(19) United States
(12) Patent Application Publication
(10) Pub. No.: US 2011/0060252 A1
(43) Pub. Date: Mar. 10, 2011

(54) Monitoring Apparatus and Method

(76) Inventors: Jan Simonsen, Struer (DK); Hanne Simonsen, Struer (DK); Michael Noel Kiernan, Swansea (GB)

(21) Appl. No.: 12/809,762

(22) PCT Filed: Dec. 19, 2008
(86) PCT No.: PCT/GB2008/051217

§ 371 Date: Aug. 30, 2010

(30) Foreign Application Priority Data
Dec. 21, 2007 (GB) 0724971.7

(51) Int. Cl. A61B 5/103

(52) U.S. Cl. 600/595

(57) ABSTRACT

The apparatus for monitoring muscle contractions of a subject (for example as a check on epilepsy or Parkinson's disease) comprises: a first sensor arranged to be placed on a reference location on the body of a subject for sensing, over a prolonged period of time, a reference parameter derived from at least one of movement and electrical activity of the muscles of the body at the reference location; at least one further sensor arranged to be placed on a chosen location on a limb of a subject, for sensing, over a prolonged period of time, a monitoring parameter derived from at least one of movement and electrical activity of the muscles of the limb at the chosen location; comparison means for comparing the monitoring parameter with the reference parameter; and output means for producing an output by comparison of the monitoring parameter and the reference parameter, the output comprising either recorded data which provide a record of an epileptic event in the prolonged period of time or an alarm output after the prolonged period of time when the monitoring parameter is outside a predetermined range compared to the reference parameter.

### BACKGROUND OF THE INVENTION

1. Field of the Invention

The present invention relates to methods and apparatus for monitoring persons suffering from (or believed to be suffering from) epilepsy, Parkinson's disease or the like disease, as well as conditions such as apnoea.

2. State of the Art

U.S. Pat. No. 6,095,991 describes an apparatus and methods where a so-called accelerometer is used to detect motions of a body or parts of a body to which the accelerometer is attached. It is mentioned that the accelerometer may be used to monitor persons suffering from epilepsy. The accelerometer is a mercury sensor, thus capable of monitoring epileptic seizures in relation to gravity. This may be valuable in order to monitor a person's non-intended motions or positions in relation to the ground, a chair, a bed or other stable supports normally used by a person. However, it is also normal for a person to use a car, a bus, a bicycle or other means of transportation. These means of transportation sets the person in motion; not only towards the destination of the travel; but into shaking motions as example when driving on an uneven surface. This may result in the sensor setting of an alarm. Also, other activities such as walking, running and biking may set off an alarm by the sensor, although an alarm is not intended in those situations.

DE 19817586A describes a system for monitoring movement of an arm or a leg of a person, also in connection with persons suffering from epilepsy. The system is based on the idea of the bracelets following the movements of the person and the system thereby learning when the person's movements are normal and when they relate to an epileptic seizure. Thereby, it is possible to conduct a therapy, which is directed towards the pattern of epileptic seizures which the individual person is suffering from. However, there is still no distinction between ordinary movements and movements related to the activity of the person, i.e. whether the person is travelling or is physically active in some way or the other.

WO2006/134359 describes a seizure detection apparatus, having a motion detector which is said to be sensitive to a seizure, and an alarm which is raised on detection of a seizure by the motion detector. The document discloses that a filter is used to determine whether motion detected is associated with a seizure; if if is determined that the motion is indeed associated with such a seizure, then after a predefined input, the alarm is initiated. A problem with this approach is that the system must be pre-programmed with the characteristics of a seizure, and does not take account of the fact that such characteristics vary widely from person to person.

### OBJECT OF THE INVENTION

It is the object of the present invention to provide a method and apparatus which can alleviate problems such as those described above, permitting monitoring of epileptic and other seizures, not only when the person is relatively passive (standing, seated or lying down) but also when the person is subjected to movement either as a result of physical activity or as a result of the person travelling in a vehicle or the like.

### SUMMARY OF THE INVENTION

In preferred embodiments, the method and apparatus are intended for monitoring muscle movements arising from absence seizures (petit mal seizures) of a person suffering from (or potentially suffering from) epilepsy and/or for monitoring muscle movements arising from tonic/clonic seizures (grand mal seizures) of such a person.

However, many other movements of the body may be monitored, such movements correlating to unintended movements associated with diseases or other malfunctions of the motor apparatus of a person. Although the two types of seizure are very different in their nature; the method and apparatus according to the present invention may be used to monitor one or both. In further embodiments, the method and apparatus according to the invention may be used to monitor unintended lack of movement, for example in the case of monitoring for apnoea or the like.

According to the invention there is provided a method of monitoring muscle contraction, which comprises placing a plurality of monitoring sensors each on a chosen location on a body, each of said sensors arranged to obtain a signal derived from movement and/or electrical activity of the muscles at the chosen location; comparing said signals with each other and with a reference signal obtained from one of said sensors; and producing an output when the signal from at least one of the sensors is outside a predetermined range compared to said reference signal.

The present invention further provides apparatus for carrying out the method according to the invention, the apparatus comprising a plurality of monitoring sensors suitable to be placed on chosen locations on the body, each of the sensors being arranged to obtain a signal derived from movement and/or electrical activity of the muscles of the body at the chosen location; means for comparing said signals with each other and with a reference signal obtained from one of said sensors, and means for producing an output when the signal from at least one of the sensors is outside a predetermined range compared to said reference signal.

When the method and apparatus according to the invention are used to monitor muscle movements arising from petit mal seizures or grand mal seizures, at least one of the sensors is preferably provided on a limb (arm or leg) of the patient.

Data produced by the sensors at the chosen locations may be transmitted to a central data analysis and recording unit, the latter being usually mounted on the body. The resulting data analysis may help to provide an indication of whether a person suffers from a condition such as epilepsy, Parkinson's disease or the like, and if they do, the time, duration, and intensity of any events.

In one embodiment, data received by such a central data analysis unit may be analysed in “real-time” to determine whether a seizure is taking place, and depending on the results of the analysis, a suitable audible or visual alarm activated. Alternatively, or additionally, the sensor data may be stored in a suitable memory device provided in the central data analysis unit, for evaluation at a later time.

When sensors of electrical activity are employed, they may be based upon Surface Electromyography (s-emg), and be arranged to detect electrical activity from muscles using conductive pads placed on the skin. When the muscle beneath the conductive pad is resting, there is a baseline signal; when the muscle is contracted by voluntary means, that is, using the muscle to achieve movement or other physical activity, the signal produced by the sensor changes to a certain range of amplitudes and frequencies. When the muscle is subject to involuntary activity, that is, when the body is experiencing a seizure, the signal produced by the sensor will have a different range. Both the amplitude and frequency of the electrical signal within the muscle are preferably monitored by the sensor.

When sensors of muscle movement are employed, these may be on a primary embracing attachment intended to be a tight fit round a part- of the body (such as around a part of an arm or around a part of a leg, preferably around the wrist of an arm or around the ankle). Each such primary embracing attachment may be provided with one or more sensors for monitoring movement of the relevant part of the body, and may include means for communicating a signal and comparison of the signal to a signal from at least one reference sensor on another part of the body.

In some embodiments of the invention, instead of being provided on an embracing attachment, the sensor may be on a strip provided with adhesive or the like to be secured to a body part.

The output obtained in the method and apparatus according to the invention may be an audible or visible alarm when muscle movement indicative of disease is detected (or, in the case of monitoring apnoea) when lack of muscle movement is detected. Such an alarm may be provided on the body, or remotely, as will be described subsequently with reference to preferred embodiments of the invention.

When such alarms are provided, there may be provided means for manual override or cancellation of the alarm signal when the user or other person realises that a false alarm has been generated.

The sensor used in the apparatus according to the invention may be provided with means for transmitting detected data to a remote monitoring location.

### BRIEF DESCRIPTION OF THE DRAWINGS

Preferred features of the invention will now be described further, by way of example only, with reference to the accompanying drawings.

FIG. 1 is a schematic view of the positioning of apparatus according to the invention;

FIG. 2 is a schematic view of a typical electrical sensor incorporating radio data transmission;

FIG. 3 is a schematic view of a sensor incorporating remote data transmission for use according to the invention;

FIG. 4 is a schematic view of the central data analysis unit for use according to the invention;

FIG. 5 (FIGS. 5a, 5b and 5c) is a schematic view of a possible set-up for monitoring bodily movements of a person; and

FIG. 6 is a schematic view of a possible means for detecting, storing and transmitting any data related to a possible movement of a body part resulting from a seizure when movements are to be monitored and communicated to other means.

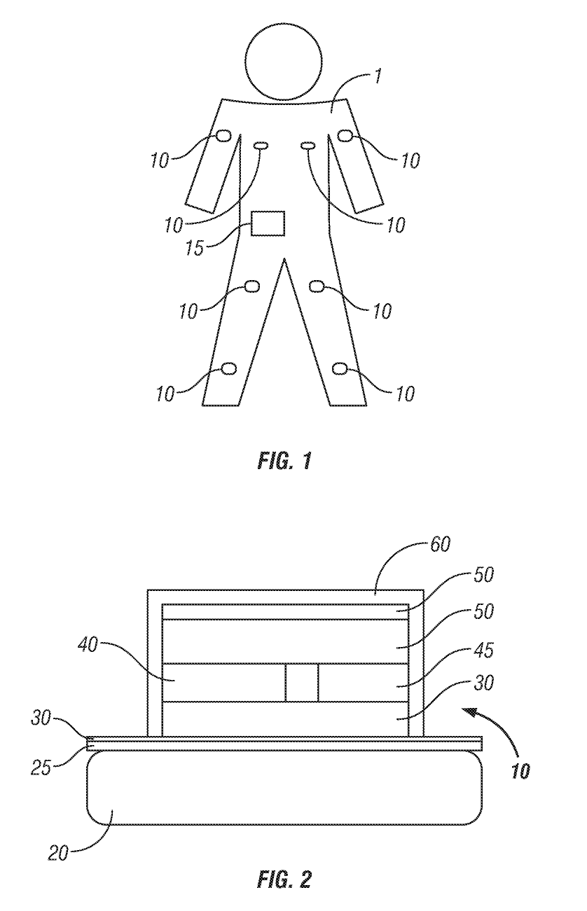

Referring to FIG. 1, sensors 10 are placed at pre-determined locations on the body 1, the locations chosen such that the sensors 10 are in contact with the skin directly over the muscle to be monitored. The number of sensors 10 may be varied but at least two sensors are required to determine activity at one location with reference to a second location. Data signals are transmitted from the sensors 10 to a central data analysis unit 15.

FIG. 2 shows an example of a typical sensor 10. The sensor comprises an outer protective housing 60 that prevents access to the sensor internal structure and also protects against external contaminant, such as water, from penetrating the housing and damaging the sensor. The sensor is mounted on the skin surface 25 directly above the muscle 20 to be monitored. The sensor is held to the skin surface 25 by a suitable adhesive layer 30; such an adhesive layer 30 must provide both mechanical attachment of the sensor 10 to the skin surface 25 and good electrical contact between the electrode 35 and the skin 25.

The electrode 35 is used to measure the electrical activity of the muscle 20 underneath the skin surface 25. There are several different electrode types but in general they consist of a strip of a suitable metal, typically silver or chemical grade steel. Good electrical contact is necessary between the electrode 35 and the skin surface 25. The adhesive layer 30 is manufactured such that the electrode is in direct contact with the skin surface 25 through a suitable aperture in the adhesive layer 30. The electrode 35 may be passive, that is, with no internal electronics for signal processing, or active, where internal electrical circuitry provides signal amplification within the electrode structure.

In a further embodiment, a suitable material may be placed between the electrode 35 and the skin surface 25 to increase the electrical conductivity and enhance the electrical signal. This material may be a gel or paste with suitable electrical properties.

The signals from the electrode 35 are processed by sensor electronics 40. The sensor electronics 40 take the electrical signals from the electrode 35, process these signals, e.g. by amplifying the signal levels or providing electronic filtering of the signals from the electrode 35. In addition to the processing of the signals from the electrode 35, the sensor electronics 40 may convert the signals into a pre-determined format necessary for transmission to the central data processing unit 15 via a transmitter/receiver 45. The transmitter/receiver 45 transmits the signals to the central data processing unit 15 via radio frequency (RF). The incorporation of the receiver function in the transmitter/receiver unit 45 allows the central data processing unit 15 to transmit signals to the sensor 10. These signals sent by the central processing unit 15 may be used to interrogate the sensor 10 to determine whether the latter is operating correctly, or to perform a measurement of the electrical activity of the muscle 20 at a defined time point or in response to activity being recorded on a different sensor 10 on another part of the body 1.

A power source is provided via a battery 50 to provide power to drive the sensor electronics 40 and the transmitter 45.

An electrical shield 55 can be incorporated into the sensor 10 to protect the internal sensor electronics 40, the transmitter 45, or the electrodes 35 from interference from external sources of electrical noise, which might otherwise affect the signals received from the muscle 20.

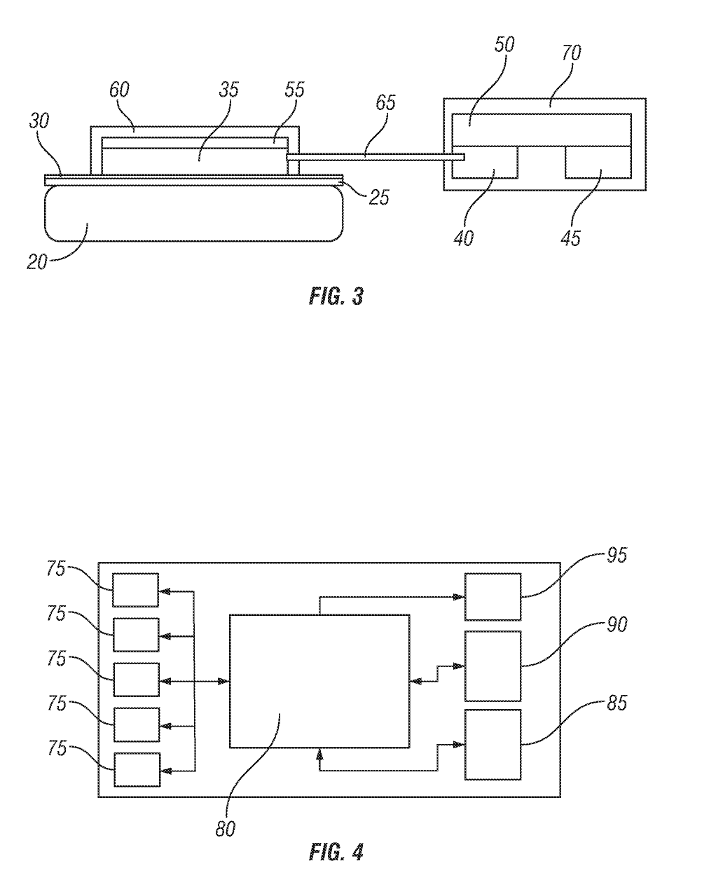

FIG. 3 shows a further embodiment of the sensor 10 which has external data transmission. The power source 50, the sensor electronics 40 and transmitter/receiver unit 45 are mounted in a separate housing 70 and the signal from the electrode 35 is transferred to the sensor electronics 40 via a suitable cable 65.

An alternative approach is to incorporate the sensor electronics 40 in the housing for the central data processing unit 15 with a direct cable connection to the sensors 10. This approach removes the requirement for the transmitter/receiver units 45. In addition, the power source for the sensors 10 can be located within the central data processing unit 15.

FIG. 4 shows a schematic layout of a suitable exemplary central data processing unit. Signals transmitted from the sensors 10 are received by the central data processing unit by the internal transmitter/receiver units 75. If the sensor type used does not incorporate an RF transmitter and instead uses a direct cable connection, the transmitter/receiver units 75 will be replaced by sensor electronics 40.

The data from the transmitter/receiver units may be transferred to the microprocessor 80 for analysis. The microprocessor 80 is arranged to analyse the signals and under predetermined conditions, either to store the information in the memory unit 90 for later use or to provide an alarm when a seizure condition is encountered. The alarm 95 may be audible or visual.

If the data is stored for later analysis, the microprocessor 80 should incorporate a real-time clock so that the timing of any events monitored by the sensors 10 can be recorded and stored in the memory unit 90.

Stored data can be transferred to a remote data analysis unit via the data transfer unit 85. The data transfer unit may be either based upon RF or a suitable electrical connector. The stored data can be analysed to determine the frequency and timing of seizures, the intensity of seizures and, if applicable, whether or not prescribed treatment is impacting on the seizure events.

When the body is undergoing voluntary movements, the signals from the sensors are monitored by the central processing unit and recorded as a baseline. Recording of these baseline signals can take place at intervals of the order of seconds. In the event of a signal being received by the central processing unit being outside some predetermined range, that is, a signal whose amplitude and frequency is different from the baseline level, the central processing unit will take samples from other sensors at different body locations to determine if the initial event is related to a seizure event or a false positive.

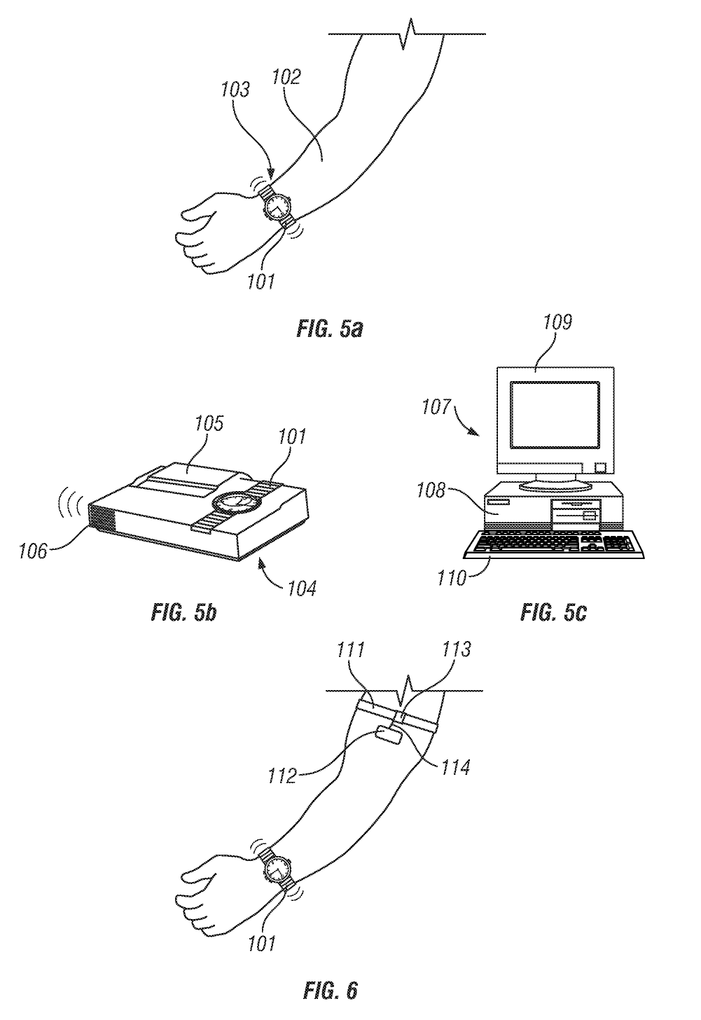

FIG. 5 shows schematically a set-up for monitoring the movement of muscles in a patient's arm and comparing with movement of other parts of the body of the patient. The patient is wearing a wriststrap 101 embracing the wrist 103 of the patient's arm 102. The wriststrap 101 may be incorporated into an ordinary wristwatch.

According to the invention, such a wriststrap 101 is provided with a sensor for sensing movement thereof relative to a reference remote from the wrist. Alternatively, the wriststrap may be provided with transmitting means for transmitting signals to a receiver either on another part of the body of the patient, or remote from the body of the patient.

A reference sensor, in relation to which the movement of the arm is compared, may be placed on one or more other parts of the patient's body, preferably on the torso of the patient, and the receiver, if provided, may also be placed on another part of the body. Preferably, both the reference sensor and the receiver are placed in a belt or other garment, such as an ordinary garment for the patient. Thus, with a sensor and transmitter provided on a wriststrap, a reference sensor provided on another part of the body of the patient and preferably in a belt, and a receiver also provided in the belt, all parts of the apparatus, which are to be worn by the patient, can be provided in ordinary garments of the patient's choosing. This can ensure that no part of the apparatus is easily visible so there is no outward indication that the patient may have a disease.

The sensor and the transmitter, if provided, on such a wriststrap are preferably passive, that is, the measurements made and the possible signals transmitted may be either continuous or only initiated when a certain pattern of movement of the muscle has been detected. Thus, either the sensor or the transmitter, if provided, is not initiated just by movement of the body part; which might otherwise be falsely identified as a seizure.

The receiver in the belt or other garment is, however, preferably active and is capable of detecting, when the passive signals from the transmitter are of such nature that a seizure of the patient's body part (such as the arm) is taking place, by comparison with a signal obtained from another body part and a reference.

The signals transmitted from the sensor in the wriststrap differ in amplitude, frequency or distance when a seizure is taking place. This is detected by the receiver. However, the receiver is preferably only initiated when signals transmitted are detected as being transmitted due to a seizure. Having both or at least one of the transmitter and receiver, respectively, only being initiated when a seizure actually is detected, then the consumption of electrical energy of the transmitter and/or the receiver may be limited.

Apart from detecting the amplitude, the frequency and/or the relative distance between the transmitter and the receiver, the receiver may also detect whether the patient is standing, sitting or lying down. This may be done by a measuring of the angular inclination of the receiver knowing that a certain inclination means that the patient is lying down. Most seizures, especially tonic/clonic seizures, result in the person lying down and this may be an indication, at least during daytime hours, that a seizure is taking place.

An alternative way of detecting whether or not a seizure is present is to use transponders instead of a transmitter in the wriststrap or the like. The receiver initially sends a signal to the transponder, and the transponder identifies itself by a coding dedicated to the transponder. The transponder should only identify itself if a seizure is taking place, and should not respond to the signal from the receiver if a seizure is not detected. The frequency of the signals transmitted by the transmitter from the wriststrap to the receiver is preferably below 9 kHz. This frequency is the limit under which approval from proper authorities is not necessary. Although signals with frequencies below 9 kHz limits the transmission range of the signals, in the present invention this does not matter. Alternatively, newly introduced or not yet introduced standards of wireless communication sensors on patients may use signals having frequencies in the 8 kHz range.

Instead of, or perhaps additional to, a transmitter for transmitting signals to a remote receiver on other parts of the body of the patient, the wriststrap may optionally include storage means for storing data of the seizures sensed by the sensor. Such storage means may alternatively be in a remote receiver, which would necessitate that the seizures measured are recorded by or transmitted to the remote receiver. The storing of the data should at least relate to whether on not seizures have actually taken place. Preferably, the data also relate to the vigour of the seizures and/or the duration of the seizures and/or the time of day the seizures have taken place.

In the embodiment of FIG. 5b, a recording and storing means is shown as a kind of a docking station 104 for signals transmitted from the wriststrap, the signals being based upon measurements made by a sensor in the wriststrap. Preferably, the docking station is for data stored in the sensor; the data subsequently being transmitted to the docking station. In the latter circumstance, the docking station is for measurements made and also stored in the wriststrap sensor, and the data may be transmitted to the docking station when the wriststrap device is placed in the docking station as shown. This may be convenient in the circumstances, where the transmitting range of the transmitter is such that it is out of reach of the docking station.

Alternatively, measurements made by the sensor may be transmitted to the docking station as soon as the transmitting range of the transmitter is within reach of the docking station. In the embodiment shown, the docking station is provided with a printer 105 for printing data related to measurements made by the sensor and transmitted to the docking station. Thereby, the patient himself or herself, or relatives or other personnel surveying the patient, may survey the illness and take proper action if the illness develops (such as sending for a doctor or an ambulance).

Apart from means for receiving and storing the data from the sensor, the docking station may include means for generating reports utilising the printer, and means for communicating with the sensor, for example, to erase the memory of the storage means of the sensor as soon as the docking station has received and stored the data transmitted from the sensor. The docking station may also comprise a loudspeaker 106 for alarming personnel in the vicinity of the docking station, if a signal from the wriststrap sensor of a seizure being detected is transmitted directly to the docking station.

The docking station (or other means for transmitting sensed data) may be provided with an internet port so that data can be transmitted over the internet for remote monitoring.

Furthermore, the docking station may be provided with a battery and optionally also a battery indicator. Thereby, the docking station may be free from the need to provide a mains electrical power supply, and/or may have a back-up power supply in case of electrical power cuts from a public network.

The alarm function of the docking station is preferably employed when the transmitter, if provided, transmits a signal because of a seizure being detected. Personnel, such as relatives to the patient, in the vicinity of the docking station are thereby alarmed. Such alarming of personnel in the vicinity of the docking station is preferably for night-time use but may also be for daytime use, if the patient is within transmitting range and other personnel are within audible range, respectively, of the docking station.

Finally, FIG. 5c shows a data processor 107 comprising a computer 108, a display 109 and a keyboard 110. The data processor 107 may store a large amount of data, and/or process the data for further use and statistical analysis. Any desired or necessary processing of the data from the docking station may be accomplished by the computer 108. Also it is possible to enable forwarding the data, either processed or not, to authorities or other bodies, which need the data, either for statistics or for medication or other treatment of the patient.

Preferably, the data processor is installed at a medical establishment, which the patient consults during control, treatment in general, and possible medication. A standardised software tool may be provided, which is capable of storing data related to the seizures measured by the sensor on the wriststrap, and which is capable of analysing the data, perhaps by generating a calendar of seizures, in order to monitor and control the illness of the patient over a prolonged period of time. Such monitoring could be useful when deciding the need for, the amount of, and the dosage of, possible medication, or when treating the patient in general. The data may be transmitted from the docking station to the computer by a data carrier such as one or more discs, memory sticks or cards, or the like, or by means of cables provided between the docking station and the computer. Such transmission may be using a public network, or may be wireless, for example using systems such as mobile telephone networks, blue-tooth transmission or any other suitable means of wireless communication.

The arrangement shown in FIG. 5 may be applied in different circumstances. Children may wear the wriststrap or other body part encircling (embracing) means during night-time and the docking station may be placed in the bedroom of the parent or carer so that they can be alarmed if a seizure develops during the night. Epileptic persons, or other patients suffering from undesired movements of the body, but living alone, may have the opportunity to automatically call for a doctor or an ambulance by means of proper telecommunication means between the docking station and a control centre. The docking station itself may be provided with a mobile phone unit, or the transmitting means of the wriststrap or of the docking station may communicate with an ordinary mobile phone being switched on automatically calling the control centre when a seizure alarm is detected.

The docking station may be used for a plurality of patients. In this case, the docking station must be able to differentiate between signals received from the different patients. This applies especially to the situation where the docking station is used for further transmittal of the alarm, but also applies where the docking station is provided with means for storing data received from different patients.

The wriststrap sensor shown in FIG. 5 may be used to monitor movement of muscles of a patient suffering from (or potentially suffering from) epilepsy, Parkinson's disease or the like disease resulting in non-intended movements of the body. The wriststrap shown in FIG. 5 may be replaced by any other suitable attachment means, such as an ordinary garment for the patient and which embraces or surrounds a part of the body of the patient.

The wriststrap or other attachment may have one or more of several possible features; all of or just some of the features may be provided in an embracement around the body part of the patient, dependent on the type of embracement and on the need for or desire for the features available. Amongst suitable features are the following:

a. A clock, for storing/transmitting a time of seizure

b. A battery for powering the clock and measurement/storing/transmitting means

c. An accelerometer for measuring the presence of and the magnitude of a seizure

d. A microprocessor for processing data from the clock and the accelerometer memory for storing processing parameters and date related to a seizure alarm for storing and/or transmitting data related to a seizure

e. An acoustic alarm for signaling to the patient wearing the watch

f. Means for communication with a possible docking station

g An indicator of functional status, such as the settings of the different alarms

h An Indicator of battery lifetime-status, for example the expected service time remaining

I A visual display of time, that is, a timer function during measurements of seizures

j On/off button for acoustic alarm, that is enabling or disabling the acoustic alarm

k. An adjustment button for sound level of acoustic alarm

;. An on/off button for seizure alarm, i.e. enabling or disabling the seizure alarm

m An adjustor for the degree of vibration of the vibration alarm

n. An adjustor for movement sensitivity

o An adjustor for time of movement

p. An adjustor for time of annulling the alarm

q. An off button for manually annulling the alarm

When the wriststrap sensor detects a seizure (that is when the signal is outside a predetermined range relative to signals from other parts of the body), the seizure alarm watch initiates a discreet signal to the wearer that a seizure is taking place. The signal may be an acoustic alarm sounding a predetermined sound, a vibration of the wriststrap or other means for messaging to the wearer. The wearer can cancel or annul a false alarm. However, by means of an accelerometer and proper programming of the microprocessor, the number of false alarms signalled to the wearer can be minimised.

FIG. 6 shows an embodiment of an apparatus, where the wriststrap 101 is not the primary means for detecting a seizure, but a secondary means. In the embodiment of FIG. 6, the wriststrap sensor communicates directly to the patient and/or transmits signals to a remote receiver that a seizure is taking place or is about to evolve. The primary detector is a relatively small embracement 111 in the shape of a strapping or plaster provided with a piezo-electrical crystal 112 capable of detecting even very small movements of the body part to which the strapping or plaster 111 is attached.

The strapping or plaster 111 is provided with a transmitter 113, and the wriststrap 101 is provided with a receiver (not shown) for receiving signals sent by the transmitter 113, the signals generated based on movements measured by the piezo-electrical crystal 112. In the embodiment shown, the piezo-electrical crystal 112 is attached to a resilient band 114, which as shown may be strapped around, for example, the upper arm of a patient. The resilient band 114 is provided with the transmitter 113, and the piezo-electrical crystal 112 is connected to the transmitter 113 via cable 115. It will be possible to manufacture the piezo-electrical crystal for daily use or perhaps even for one-time use, but to manufacture the resilient band and the transmitter for continuous use or at least several times of usage. The distance between the piezo-electrical crystal and the transmitter along the cord may vary.

The strapping or plaster 111 has the advantage compared to, for example, a wriststrap that it can be employed almost anywhere on the body of a patient, permitting detection of seizures at a wider range of locations. It may be advantageous to apply the piezo-electrical crystal to those parts of the body, where it is known that seizures may originate in the particular patient in question. Thereby, it will be possible to detect a seizure at an early stage and thus avoid discomfort, which might otherwise result from a later detection of a seizure. Also, it will be possible to limit the vigour of the seizure, if proper precautions, either medication or physical treatment of the patient, can be initiated at an early stage of the seizure.

Features of the invention which have been described with reference to the movement sensor may also be provided on the apparatus employing electrical sensors and vice versa.

1-17. (canceled)

18. Apparatus for monitoring muscle contractions of a subject, which
(a) a reference sensor having at least one conductive pad arranged to be placed on the skin of a body at a reference location for sensing, over a prolonged period of time, reference Surface Electromyography (s-emg) signals of the skin at the reference location;
(b) at least one monitoring sensor having at least one conductive pad arranged to be placed on the skin of a respective limb of the body at a monitoring location for sensing, over said prolonged period of time, monitoring Surface Electromyography (s-emg) signals of the skin at the monitoring location;
(c) attachment means for attaching said at least one monitoring sensor to the respective limb;
(d) comparison means for comparing said reference s-emg signals with said monitoring s-emg signals; and
(e) output means for producing an output by comparison of said reference s-emg signals and said monitoring s-emg signals, said output comprising either recorded data which provide a record of an epileptic event in said prolonged period of time or an alarm output after said prolonged period of time when a parameter of said monitoring s-emg signals is outside a predetermined range compared to a parameter of said reference s-emg signals.

19. Apparatus according to claim 18, wherein:
the reference sensor is arranged to transmit said reference signals; and
the monitoring sensor is arranged to transmit said monitoring signals and said

20. Apparatus according to claim 19, further comprising:
a plurality of said monitoring sensors and of said comparison means.

21. Apparatus according to claim 18, wherein:
said attachment means is arranged to be a tight fit round the respective limb.

22. Apparatus according to claim 18, further comprising:
transmission means for transmitting data from each said sensor, a receiver to be mounted on the body for receiving said transmitted data, and a data analysis unit to be mounted on the body for analyzing the received data.

23. Apparatus according to claim 22, wherein:
said output means comprises an alarm arranged to be activated depending on an output from said data analysis unit.

24. Apparatus according to claim 22, further comprises:
memory means provided in the data analysis unit for storing said received data.

25. Apparatus according to claim 18, wherein:
at least one of said sensors is on a primary embracing attachment intended to be a tight fit round a part of the body.

26. A method of monitoring muscle contraction, comprising:
(a) placing a reference sensor on a reference location on a body of a subject
(b) placing at least one monitoring sensor on a respective limb of said subject
(c) comparing the reference signals with the monitoring signals; and
(d) producing an output when monitoring signals are outside a

27. A method according to claim 26, wherein:
data produced by the sensors is transmitted to a central data analysis unit mounted on the body.

28. A method according to claim 27, wherein:
an alarm is activated depending on an output from said data analysis unit.

29. A method according to claim 27, wherein:
data obtained from the sensors is stored in the memory device provided in a central data analysis unit.

30. A method according to claim 27, wherein:
at least some of the sensors are on a primary embracing attachment intended to be a tight fit round a part of the subject.

31. A method according to claim 30, wherein:
the primary embracing attachment is provided with one or more of said monitoring sensors.
